(19) United States
(12) Patent Application Publication
(10) Pub. No.: US 2014/0171794 A1
(43) Pub. Date: Jun. 19, 2014

(54) Method of a Liquid Surgery

(71) Applicant: James Liu, Downingtown, PA (US)

(72) Inventor: James Liu, Downingtown, PA (US)

(21) Appl. No.: 13/714,352

(22) Filed: Dec. 13, 2012

(51) Int. Cl. A61M 31/00

(52) U.S. Cl. CPC A61M 31/00; A61M 31/005
USPC 600/431; 604/514

(57) ABSTRACT

A method of a liquid surgery is described. The method may include introducing a cleansing solution, a diagnostic solution or therapeutic solution into the surface of the entire upper airway with a hand-operated pump. The liquid surgery can remove viruses, bacteria, fungi, allergens, and other harmful substance from the upper airway. The liquid surgery can help diagnose or treat a disease of the upper airway.

### BACKGROUND

The present method relates generally to a non-invasive surgical procedure, devices, and more particularly to a hand operated pump to deliver a liquid knife to clean out these harmful substances from the entire upper respiratory tract, or upper airway to treat or diagnose a disease. A surgeon can remove a solid tumor locally in most part of the body with a metal knife. No surgeon, however, can use a metal knife to remove these tiny microbes, such as viruses, from the infected local area non-invasively. The surgeon cannot see these microorganisms.

The common cold is caused by any of more than 200 rhinoviruses. These viruses prefer growing at a lower temperature (33° C. or 91° F.). Therefore, the cold is a local disease in the upper airway, mainly, the nose. To cure a cold, we need to remove them physically. For this purpose, the metal knife is useless. The liquid knife, then, is the right tool.

Nasal allergy is caused by any of these pollens in the air. These tiny particles are inhaled into the upper respiratory tract. Therefore, nasal allergy is caused by the substance locally in the upper respiratory tract. To prevent or cure nasal allergy, we need to remove them physically. For this purpose, again, the metal knife is useless. The liquid knife, then, is the right tool.

Nasal spray with saline, or hypertonic saline, sometimes also called as a way of nasal irrigation, cannot be used as for nasal liquid surgery. Nasal spray was studied for its effectiveness to treat a common cold in 143 adult patients with a cold or sinus infection. Patients with allergic rhinitis, symptoms for more than 3 weeks, or other respiratory diagnoses were excluded, as were those who had used topical decongestants. No difference was found in the primary outcome—“Do you feel back to normal” after the subjects completed a 7-day symptom checklist (Adam 1998). Obviously, nasal spray can only moisturize the nasal cavity, cannot remove cold viruses. Its lack of effective is no surprise.

Neti pot has been used for centuries to remove many pathogens from the upper respiratory tract. Currently, many types of neti pots are available in the marketplace. It is a fact that neti pot delivers the cleansing liquid through natural gravity. The liquid can only run through left and right nostrils after passing through nasopharynx in the middle when one side of the nostril is at the higher position than the opposite. Oropharynx and throat cannot be cleansed by using a neti pot. These viruses in oropharynx and throat can continually grow to sustain the infection.

A significant number of people in the general population have the nasal blockage, either physically, anatomically or functionally during an inflammation. For these people, the liquid delivered from a neti pot cannot run through from the left to the right nostril. For this, neti pot is useless. It was reported that seven percent of newborn babies suffer significant nasal injury in the birth process. Nasal injuries are common in both children and adults. If these nasal injuries obstruct breathing, surgical correction will be needed before a neti pot can be used.

Some nasal irrigators are powered with electricity, either with plug in or a battery. It is a fact that the user cannot control the force and the flow rate/speed of the liquid. It is very hard for the user to cleanse the surface of the entire upper respiratory tract. Virtually all user manuals of these electronic irrigators are for rinsing the left and right nostrils and nasopharynx in the middle. Oropharynx, throat and mouth are not taught to cleanse. These parts of the upper airway harbor a variety of viruses, bacteria and other microbes. These viruses can continue their infection since these parts are not cleansed. Thus, electronic irrigators are not the right tool for the liquid surgery to clean completely these infectious microbes.

Some squeeze-bottle nasal irrigators are also available. Only one of them has the liquid-air dual-valve system invented by the current inventor (Liu, 2001 U.S. Pat. No. 6,238,377; Liu, 2004, U.S. Pat. No. 6,736.792) to enable the user to have a continual nasal irrigation without backwash contamination. Without a functional liquid valve, the user has to remove the irrigator from the nostril after each squeeze action. Otherwise, the dirty solution will be sucked back into the bottle. Virtually all user manuals of these squeeze-bottle irrigators are for rinsing the left and right nostrils and nasopharynx in the middle. Oropharynx, throat and mouth are not taught to cleanse. Again, these parts of the upper airway harbor variety of viruses, bacteria and other microbes. Thus, the squeeze-bottle irrigators without a liquid-air dual valves system are not the right tool for performing the liquid surgery.

Many drugs and biological agents have been studied for treating the common cold and granted patents. However, these candidates are very different from the liquid surgery method. A number of nasal irrigation products also received US patent protection but they are not suitable for performing a liquid surgery. The dual-valve nasal irrigation system is currently protected by the US patents (Liu, 2001 U.S. Pat. No. 6,238,377; Liu, 2004, U.S. Pat. No. 6,736,792). The present invention discloses a new version of the dual-valve nasal irrigation system and the method of performing a liquid surgery.

### SUMMARY

In one aspect, the invention features a method of liquid surgery. The method comprises inserting a nostril fitting of the hand-operated pump with liquid-air dual valves and with a cleansing solution. The functional solutions are: cleansing solution for the non-invasive treatment by removing these harmful substances out of the upper airway; the labeling solution to cover the area of the upper airway to add the diagnosis of a disease; the therapeutic solution with an active pharmaceutical agent to further treat a disease.

An advantage of the invention is that it provides for highly localized cleansing solution, a therapeutic or diagnostic agent without significant risk to patients. The invention is able to generate a localized cleaning force that produces a liquid knife without a significant discomfort of the patient. This substantially eliminates the risk of any damage of the nasal lining. The invention produces a self-healing opportunity. The invention permits, among other interventional procedures, localized treatments for a number of common diseases, such as common cold, nasal allergy, sinusitis, post nasal drip, etc.

The details of one or more embodiments of the invention are set forth in the accompanying drawings and the description below. Other features, objects and advantages of the invention will be apparent from the description and drawings, and from the claims.

### DESCRIPTION OF DRAWINGS

FIG. 1 is a schematic, perspective view of a hand-operated pump with both air-liquid valves on the cap. This is a new version of the previously invented nasal irrigator.

FIG. 2 is a schematic 4-step process showing how the functional liquid is moving in the upper airway:

Step 1 shows how the liquid running from the left nostril, left sinuses, nasopharynx, and right nostril.

Step 2 shows how the liquid running from the right nostril, right sinuses, nasopharynx, and left nostril.

Step 3 shows how the liquid running from the left nostril, left sinuses, nasopharynx, oropharynx, throat, and out of mouth.

Step 4 shows how the liquid running from the right nostril, right sinuses, nasopharynx, oropharynx, throat, and out of mouth.

### DETAILED DESCRIPTION

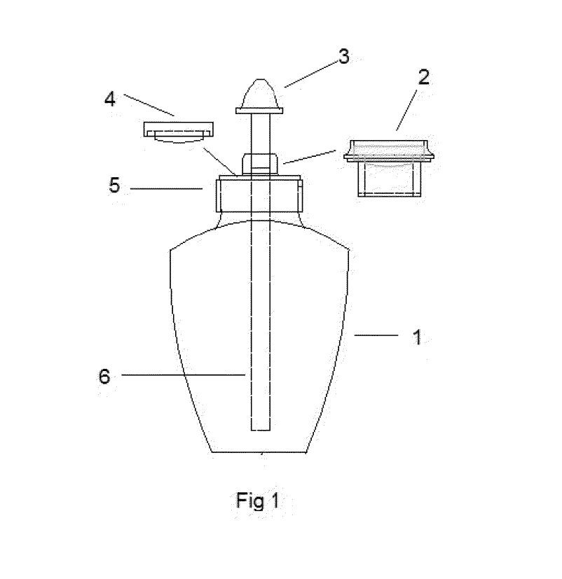

The new version of the previously invented nasal irrigation system is shown in FIG. 1. Part 1 is an elastic bottle and can be squeezed and released repeatedly. Any elastic material can be used to make this bottle. Part 2 is a one-way liquid valve in the cap. This one-way liquid valve only permits the liquid run out of the bottle, not lets the liquid back flow to the bottle. Part 3 is a one-fits-all nostril fitting. It can be removed from the cap for special cleaning or sterilization as this is the only part of the hand-pump to contact the nostril. Part 4 is an air valve which allows air only enter into the bottle to eliminate the negative pressure after the liquid is pumped out. Part 2 and Part 4 can be made as one piece with a specially configured design. Part 5 is a cap which seals the bottle and supports the air-liquid valves. Part 6 is s thin tube which is to supply the liquid from the bottle under a positive pressure.

The components of the solution can be varied to meet the specific needs. For comfort, not lower than 0.9% of sodium chloride is needed for any solution to be pumped into the nasal cavity. A pair of the buffering agents, citric acid and sodium citrate, are included for comfort and for maintaining the right pH. Other components, such as moistening agents, anti-inflammatory agents, anti-histamine agents, anti-microbial agents, labeling agents, other therapeutic agents, can be included.

This is a new version of the previously invented nasal irrigator and has several important advantages over the other nasal irrigation devices: (1) it has a pair of one-way air-liquid valves which permits the fluid to flow continually only from the suction tube into the nasal cavities without creating negative pressure within the bottle; (2) it has an one-fits-all nostril fitting, (3) the nostril fitting is exchangeable and is connected directly to the liquid valve; (4) the bottle is reusable, and serves as a blender of the pre-mix with water; (5) pumping power is supplied by the user's hand—the user can repeat gently the squeezing-releasing action to force the functional liquid to be a liquid knife to remove these harmful substance out of the upper airway; (6) the user can perform this self-cleansing liquid surgery by standing straight in front of a sink without tilt the head side-way so the chance of the liquid to run to the lower side of the ear (necessary for using a neti pot) is avoided; (7) the cleansing solution is scientifically formulated with citric acid and sodium citrate to improve the sense of smell without causing discomfort; (8) it is safe, easy to assemble, user-friendly, light, portable, durable, and inexpensive. It is easy to get the registration with the US FDA as an over-the-counter (OTC) medical device. This medical device can be used on a daily basis to treat or prevent common colds because of its safety and easiness for use.

Many labeling agents are commercially available from the current market. However, these agents have not been used to largely cover the entire upper airway for diagnosis of a disease. These agents are in different colors, tastes, and in physical, chemical or radiation properties. Now these agents, if no harm to the upper airway, can be used to touch the entire surface of the upper airway via the new liquid surgery procedure. IN the prior arts, one can easily find many markers, such as an antibody marker, a color marker, an enzyme marker, a contrast medium, a fluorescent marker, or a radioactive marker.

Many therapeutic agents are commercially available from the current market. However, they have not been used to cover the entire surface of the upper airway. These agents can be any of an inorganic pharmacological agent; an organic pharmacological agent; a biological agent; a plant extract; a cell with a treatment function including an undifferentiated, partially differentiated, or fully differentiated stem cell, an islet cell, or a genetically altered cell; and an organic genetic material including a gene, a chromosome, a plasmid, DNA, RNA, mRNA, rRNA, tRNA, synthetic RNA, or synthetic DNA.

Using the hand-operated pump with an one-fits-all nostril fitting and a dual valve mean, and containing a functional liquid, such as a cleansing liquid, a labeling liquid, a therapeutic agent, one can perform a liquid surgery to treat a disease, such as cure the common cold by completely removing/cleansing these cold viruses from the upper airway, several times a day for a quick recovery effect, without any noticeable side effects.

Below is the detail method of performing the new liquid surgery by a user:

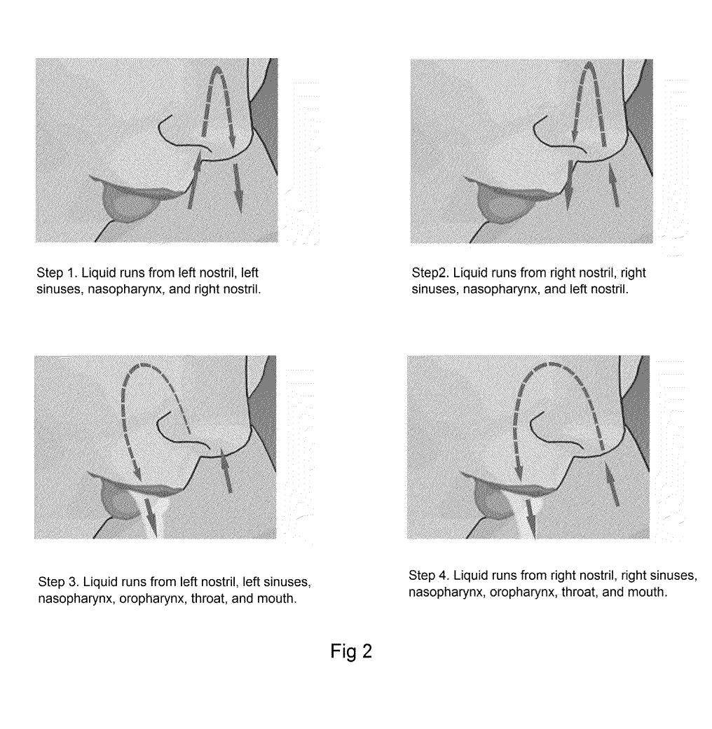

Step 1. Performing the liquid surgery to clean Part 1 of the upper airway, as shown in FIG. 2, Step 1: make a solution Next; insert the nostril fitting first to the left nostril, gently squeeze and release the elastic bottle, the functional liquid will be forced run from the left nostril, to left sinuses, to nasopharynx, and finally out of the right nostril. The liquid knife (60 ml or 2 oz) is formed inside of this part of the upper airway and physically cleans the surface of the left nostril, left sinuses, nasopharynx, and out of the right nostril.

Step 2. Performing the liquid surgery to clean Part 2 of the upper airway, as shown in FIG. 2, Step 2: make a solution; insert the nostril fitting to the right nostril, gently squeeze and release the elastic bottle, the functional liquid will be forced run from the right nostril, right sinuses, nasopharynx, and out of the left nostril. The liquid knife (60 ml or 2 oz) is formed inside of this part of the upper airway and physically cleans the surface of the right nostril, right sinuses, nasopharynx, and out of the left nostril.

Step 3. Performing the liquid surgery to clean Part 3 of the upper airway, as shown in FIG. 2, Step 3: make a solution; insert the nostril fitting tightly to the left nostril, then the user gently repeats the action of squeezing-releasing the dual-valve pump, the liquid will run to the left nostril, the liquid knife (60 ml or 2 oz) is formed inside of this part of the upper airway. When the left side of the nasal cavity is full, the user gently sniffs the liquid so the solution runs through left side sinuses, nasopharynx, left side of the oropharynx and throat, and out of mouth. This will cleanse the left nostril, left sinuses, nasopharynx, left side of the oropharynx, throat and mouth.

Step 4. Performing the liquid surgery to clean Part 3 of the upper airway, as shown in FIG. 2, Step 4: make a solution; insert the nostril fitting tightly to the left nostril, then the user gently repeats the action of squeezing-releasing the dual-valve pump, the liquid will run to the right side nostril, right sinuses. The liquid knife (60 ml or 2 oz) is formed inside of this part of the upper airway. When the right side of the nasal cavity is full, the user sniffs the liquid so the solution runs to the right sinuses, nasopharynx, right side of the oropharynx and throat, and out of mouth. Spit the solution into the sink. This will clean the right nostril, right sinuses, nasopharynx, right side of the oropharynx and throat.

This 4-step liquid surgery procedure is very safe and easy, and can be repeated every 2, 4 or 6 hours depending on how quickly the user wants to clean out these cold viruses or allergens. Since the surface of the upper airway is completely cleansed each time with 240 ml (8 oz) liquid knife, the viruses will lose the chance/time to infect the other cells. Hence, the infection is abolished. These inflammatory mediators are also removed. The symptoms, such as the common cold, therefore, will be quickly reduced.

Now the regular user can perform a liquid surgery, without hurt any tissue, to treat a number of diseases of the upper respiratory tract.

A number of embodiments of the invention have been described. Nevertheless, it will be understood that various modifications may be made without departing from the spirit and scope of the invention.

Below are several examples of using the liquid surgery to cure the common cold.

### EXAMPLE 1

A male adult had a common cold at about 3 p.m. on one afternoon. He clearly knew that he had the cold symptoms. He took one set of the hand-operated nasal pump irrigator and performed a 4-step nasal liquid surgery procedure. His sore throat, cough and sneezing seemed slightly less after the first time of removing the cold viruses. At 7 p.m. that evening, shortly after he finished his supper, he noticed his cold symptoms were still evident as mucus was streaming of his nose. He performed another complete nasal liquid surgery to cleanse out these remaining cold viruses. Before he went to bed at 11 p.m., he performed another complete nasal liquid surgery to deeply cleanse his upper airway. His nose, sinuses, nasopharynx, oropharynx, and throat cleared and he slept well that night. He did not have any noticeable cold symptoms when he woke up the next morning and he had a productive workday. The nasal liquid surgery is very effective to treat his common cold.

EXAMPLE 2

A high school student who regularly practice tennis. One afternoon after returned home, he started to cough and sneeze. His father told him that he should do a complete nasal cleansing with a hand pump nasal irrigator. He did nasal cleansing immediately, and repeated it two more times that night during home work breaks. The next morning, he drove himself to a neighborhood high school to take the national ACT exam. He did not have a headache or any other cold symptoms during the hours-long test. After he completed his test, he returned to the tennis court to practice.

EXAMPLE 3

A mother who has two young daughters became sick after her daughter had similar symptoms the two days previously during the first week of the new semester. It was no surprise to her since many school kids were infected with cold viruses soon after they started the new school year. When she was advised to perform a complete nasal cleanse to cure the cold, she with a try-and-see mind performed the complete nasal cleanse. After completing the first nasal cleanse, she felt air passing through her nose much easier than before, and she had less sneezing and coughing. Encouraged by this positive change, she performed nasal cleanse three more times that day. By the next day, her cold symptoms were gone.

| | | |
|---|---|---|
| 6,238,377 | May 2001 | Liu |
| 6,736,792 | May 2004 | Liu |
| 8,146,587 | April 2012 | Flickinger, et al. |
| 8,162,921 | April 2012 | Flickinger, et al. |
| 8,048,023 | November 2011 | Hoke, et al. |
| 8,003,688 | August 2011 | Berg |
| 7,771,757 | August 2010 | Tigunait, et al. |
| 6,361,521 | March 2002 | Erickson |
| 7,652,030 | January 2010 | Moesgaard, et al. |
| 7,166,640 | January 2007 | Berg |
| 6,641,801 | November 2003 | Brown |
| 6,492,350 | December 2002 | Konno, et al. |
| 6,380,201 | April 2002 | Johnson, et al. |
| 5,962,473 | October 1999 | Johnson, et al. |
| 5,890,486 | April 1999 | Mitra, et al. |
| 5,869,497 | February 1999 | Jolinson, et al. |
| 5,626,831 | May 1997 | Moerkerken |
| 5,622,724 | April 1997 | Bryce-Smith |
| 4,956,385 | September 1990 | Eby, III |
| 5,899,878 | May 4, 1999 | Glassman |

### OTHER REFERENCES

Adam P, Stiffman M, Blake R L Jr. A clinical trial of hypertonic saline nasal spray in subjects with the common cold or rhinosinusitis. Archives of Family Medicine 1998, 7(1):39-43.

McCaffrey T V and Kern E B. Clinical Evaluation of Nasal Obstruction, A Study of 1,000 Patients. Arch Otolaryngol 1979 105:542-545.

What is claimed is:

1. A method of liquid surgery comprising: (1) having a hand-operated nasal pump containing a cleansing solution, (2) inserting a nostril fitting of the pump tightly into one nostril and then gently pump to force the liquid run from that side of nostril to that side of sinuses, to nasopharynx, and out of the other side of nostril; (3) inserting a nostril fitting of the pump tightly into one nostril and then gently pump to force the liquid run from that side of nostril, to that side of sinuses, nasopharynx, oropharynx, throat, and mouth, then spit the solution to a sink

2. A method of liquid surgery of claim 1 whereas for these patients whose left and right nostrils are not connected can take an alternative procedure comprising: (1) having a hand-operated nasal pump containing a cleansing solution, (2) inserting a nostril fitting of the pump tightly into one nostril and then gently pump to force the liquid run from that side of nostril, to that side of sinuses, nasopharynx, oropharynx, throat, and mouth, then spit the solution to a sink.

3. A method of liquid surgery of claim 1 is to remove a microbial pathogen from the upper airway.

4. A method of liquid surgery of claim 1 is to remove an allergic substance from the upper airway.

5. A method of liquid surgery of claim 1 is to remove toxic substance from the upper airway.

6. A method of liquid surgery of claim 1 is to remove an inflammatory factor from the upper airway.

7. A method of liquid surgery of claim 1 is to remove excessive mucus from the upper airway.

8. A method of liquid surgery of claim 1 is to treat an upper respiratory tract infection.

9. A method of liquid surgery of claim 1 is to treat an allergic disorder related to the upper airway.

10. A method of liquid surgery of claim 1 is to treat an inflammatory disorder the upper airway.

11. A method of liquid surgery of claim 1 is to treat post nasal drip.

12. A method of liquid surgery of claim 1 is to clean the upper airway before or after a surgical operation of mouth, ear, nose or throat.

13. A method of liquid surgery of claim 1 is to clean the upper airway after a chemotherapy or radiotherapy.

14. A method of liquid surgery of claim 1 is to clean the upper airway after a radiotherapy.

15. A method of liquid surgery comprising: (1) having a hand-operated nasal pump containing a specifically formulated solution with a diagnostic or therapeutic agent , (2) inserting a nostril fitting of the pump tightly into one nostril and then gently pump to force the specific solution run from that side of nostril to that side of sinuses, to nasopharynx, and out of the other side of nostril to cover the surface of these areas in the upper airway; (3) inserting a nostril fitting of the pump tightly into one nostril and then gently pump to force the specific solution run from that side of nostril, to that side of sinuses, nasopharynx, oropharynx, throat, and mouth to cover these areas for disease diagnosis.

16. The method of claim 15 wherein the diagnostic agent in the labeling solution is selected from the group consisting of an antibody marker, a color marker, an enzyme marker, a contrast medium, a fluorescent marker, or a radioactive marker.

17. The method of claim 15 wherein the therapeutic agent is selected from the group consisting of an inorganic pharmacological agent; an organic pharmacological agent; a biological agent; a plant extract; a cell with a treatment function including an undifferentiated, partially differentiated, or frilly differentiated stein cell, an islet cell, or a genetically altered cell; and an organic genetic material including a gene, a chromosome, a plasmid, DNA, RNA, mRNA, rRNA, tRNA, synthetic RNA, or synthetic DNA.

18. The method of a liquid surgery by using a hand-operated pump system comprising:
1) a pair of one-way liquid and air valves assembled together or made as one piece;
2) an exchangeable nostril fitting to have the function of one-fits-all sizes of nostrils;
3) an elastic bottle to be squeezed-released to form a pump pressure for a liquid;
4) a thin tube directly connecting to the liquid valve for easy assembly;
5) a basic liquid containing sodium chloride no less than 0.9%, citric acid and sodium citrate to maintain a pH of the solution from 5 to 9; and a functional agent, such as a detergent, a moistening agent, a labeling agent, a therapeutic agent.

19. The method of claim 18 wherein the liquid surgery is to treat or prevent a respiratory disease of a mammal, such as a common cold.

20. The method of claim 18 wherein the liquid surgery is to treat or prevent a systemic disease of a mammal, such as diabetes.
